(19) United States
(12) Patent Application Publication
(10) Pub. No.: US 2019/0201540 A1
(43) Pub. Date: Jul. 4, 2019

(54) IMMUNOGENS, COMPOSITIONS, AND METHODS FOR TREATING DYSLIPIDEMIA

(71) Applicants: Bryce Chackerian, Albuquerque, NM (US); Alan Remaley, Bethesda, MD (US); Ingrid Lindquist, Albuquerque, NM (US); Ingrid Lindquist, Albuquerque, NM (US); STC UNM, Albuquerque, NM (US)

(72) Inventors: Bryce Chackerian, Albuquerque, NM (US); Alan Ramaley, Bethesda, MD (US); Ingrid Lindquist, Albuquerque, NM (US)

(21) Appl. No.: 16/304,020

(22) PCT Filed: May 24, 2017
(86) PCT No.: PCT/US2017/034149

§ 371 Date: Nov. 21, 2018

(51) Int. Cl. A61K 47/64; A61K 39/00; A61K 39/12; A61K 47/69; C07K 14/775; A61P 3/06; C12N 15/85

(52) U.S. Cl. CPC A61K 47/646; A61K 39/0005; A61K 39/12; A61K 47/6901; A61K 45/06; A61P 3/06; C12N 15/85; A61K 2039/62; C07K 14/775

(57) ABSTRACT

This disclosure describes immunogens, compositions, and methods for treating dyslipidemia. The immunogen included an ApoC3-derived peptide linked to a bacteriophage virus like particle (VLP) immunogenic carrier. The ApoC3 immunogen can be administered to a subject having, or at risk of having, dyslipidemia. The ApoC3 immunogen may be administered alone or co-administered with an additional dyslipidemia therapeutic agent.

### CROSS-REFERENCE TO RELATED APPLICATION

This application claims priority to U.S. Provisional Patent Application No. 62/340,706, filed May 24, 2016, which is incorporated herein by reference.

### SEQUENCE LISTING

This application contains a Sequence Listing electronically submitted to the United States Patent and Trademark Office via EFS-Web as an ASCII text file entitled “2017-05-24-31001090201-SEQUENCE-LISTING_ST25.txt” having a size of 2.21 kilobytes and created on May 24, 2017. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes, in one aspect, an ApoC3 immunogen that includes an antigenic ApoC3 peptide linked to a bacteriophage virus like particle (VLP) immunogenic carrier. In some embodiments, the antigenic ApoC3 peptide includes the amino acid sequence STVKDKFSEF (SEQ ID NO:2). In some embodiments, the VLP immunogenic carrier can include Qβ.

In another aspect, this disclosure describes an immunogenic composition that includes any embodiment of the ApoC3 immunogen and an adjuvant.

In another aspect, this disclosure describes a method of treating a subject having, or at risk of having, dyslipidemia. Generally, the method includes administering to the subject an effective amount of any embodiment of the ApoC3 immunogen. In some embodiments, the method can further include co-administering to the subject at least one additional dyslipidemia therapeutic agent. In some of these embodiments, the additional dyslipidemia therapeutic agent can include a statin, a bile acid sequestrant, niacin, a fibric acid derivative, or a long chain alpha, omega-dicarboxylic acid.

The above summary is not intended to describe each disclos [symbol] implementation of the present invention. The description that follow [symbol] exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. This figure shows the conjugation of ApoC3 peptides (SEQ ID NO:1 and SEQ ID NO:2) to Qβ bacteriophage VLPs using a bifunctional cross-linker.

FIG. 2. Antibody titers in immunized mice. Groups of mice were immunized with the indicated VLPs. Sera were tested for antibody responses against either full-length ApoC3 (filled circles), the a2 peptide (open circles), or the a5 peptide (triangles). End-point dilution IgG antibody titers are shown. Each data point indicates an individual mouse.

FIG. 3. (A) Triglyceride levels in immunized mice. (B) Total cholesterol levels in immunized mice. Groups of eight mice were immunized with the indicated VLPs. Plasma lipids levels were measured prior to immunization (black bars) and two weeks after the second immunization (white bars). Bars show the mean values plus the standard deviation. * indicates p<0.05, by 2-way ANOVA.

FIG. 4. ApoC3 levels in the sera of immunized mice, as determined by ELISA. Each data point represents an individual mouse. * indicates p<0.05 by Dunnett's multiple comparisons test.

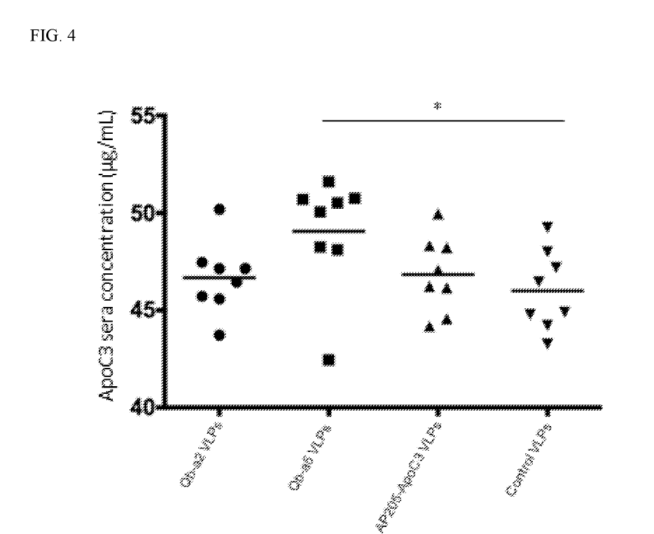

### DETAILED DESCRIPTION OF ILLUSTRATIVE EMBODIMENTS

This disclosure describes immunogens, compositions, and methods for treating dyslipidemia. The immunogen included an ApoC3-derived peptide linked to a bacteriophage virus like particle (VLP) immunogenic carrier. The ApoC3 immunogen can be administered to a subject having, or at risk of having, dyslipidemia. The ApoC3 immunogen may be administered alone or co-administered with an additional dyslipidemia therapeutic agent.

Dyslipidemia is a risk factor for the development of cardiovascular disease, specifically coronary artery disease. A harmful, atherogenic lipid profile includes elevated serum levels of low-density lipoprotein cholesterol (LDL-C), low serum levels of hi [symbol] cholesterol (HDL-C), and elevated serum triglycerides (TG). LDL- [symbol] molecules—i.e., they tend to promote the formation of fatty plaques in arteries. Their role in the pathophysiology of cardiovascular disease is well understood and LDL-Cs are the therapeutic target of certain drugs, including HMG-CoA reductase inhibitors (statins). Although lowering LDL-C reduces cardiovascular risk, it does not completely eliminate it; up to 19% of those with therapeutically optimized LDL-C levels still go on to have a cardiovascular event. Improving levels of HDL also is a therapeutic goal as these molecules transport LDL from peripheral vessels to the liver and can, at higher levels, reduce LDL-C in the serum. Moreover, high serum triglycerides independently indicate an increased risk of, for example, cardiovascular disease, diabetes, metabolic syndrome, and obesity. Thus, medical management of serum triglycerides decrease the likelihood and/or extent of cardiovascular disease.

Triglycerides are molecules that store energy in the body. Triglycerides are synthesized in the liver and can also be absorbed in the intestine from the diet. They are transported in blood vessels by triglyceride rich lipoproteins (TRLs) including, for example, very low-density lipoproteins (VLDL) and chylomicrons. These, and other lipoproteins, contain apolipoproteins on their surface, which help to stabilize and mediate transport dynamics. One of the apolipoproteins that associates with triglyceride rich lipoproteins is apolipoprotein C III (ApoC3). Mature ApoC3 is a 79 amino acid glycoprotein. ApoC3 inhibits the enzyme lipoprotein lipase (LPL), which normally hydrolyses the TRL, creating free fatty acids (FFA) that can be taken up by target muscle tissues and used for energy or reorganized into triglycerides again. Inhibition of LPL (and hepatic lipase activity) delays the metabolism and clearance of triglycerides and LDLs. ApoC3 can directly initiate endothelial dysfunction including, for example, direct adhesion activation, proinflammatory activation, and inhibition of NO production. ApoC3 also may regulate an LPL-independent clearance of triglycerides.

Mutations in ApoC3 can affect TG levels. For example, ApoC3 knockout mice are hypotriglyceridemic, whereas overexpression of ApoC3 leads to hypertriglyceridemia. In humans, the findings are similar. Increased ApoC3 levels are an independent risk factor for coronary heart disease. Conversely, loss-of-function genetic variations in the ApoC3 gene that have been identified in several isolated populations are associated with low TGs, high HDL-C, and low LDL-C levels. Follow-up studies on larger population cohorts found that loss-of-function ApoC3 variants were associated with reduced risk of ische [symbol] In a recent exome-sequencing project of 3,734 individuals, low trigl [symbol] associated with mutations in ApoC3. One quarter of the subjects were found to be a heterozygote in at least one of four common dysfunctional variants of ApoC3. Individuals with these loss-of-function mutations had statistically significant cardiovascular disease protection, including 39% lower plasma triglyceride levels, 22% higher HDL cholesterol levels, 16% lower LDL cholesterol levels, and a 40% reduction of coronary heart disease.

An antisense oligonucleotide targeting ApoC3 mRNA (ISIS 304801, volanesorsen, Ionis Pharmaceuticals, Inc., Carlsbad, Calif.) has been evaluated in a phase 2 clinical trial in patients with hypertriglyceridemia. Administration of volanesorsen was associated with an 80% reduction in ApoC3 levels and a 70% reduction in TG levels. No safety concerns were identified in the trial.

This disclosure describes the design and engineering of three VLP-based vaccines to target ApoC3. Displaying a target antigen on the surface of a virus-like particle (VLP) can increase the immunogenicity of the antigen. VLP display can elicit strong antibody responses against self-antigens, which are normally poorly immunogenic. ApoC3 peptides were displayed on bacteriophage VLPs either by conjugating synthetic peptides to Qβ bacteriophage VLPs (Jegerlehner et al., 2002, Vaccine 20:3104-3112) or by constructing a recombinant AP205 bacteriophage VLP displaying full-length ApoC3 (Tissot et al., 2010, PLoS One 5(3):e9809). For chemical conjugation, peptides representing human ApoC3 amino acids 22-30 (a2 peptide; TAKDALSSV (SEQ ID NO:1) and 55-64 (a5 peptide; STVKDKFSEF (SEQ ID NO:2)) were synthesized with a C-terminal GGGC tag to allow conjugation to Qβ VLPs. Conjugations were performed as previously described (Chackerian et al., 2006, Vaccine 24:6321-6331) and resulted in display of ˜300 peptides per VLP (data not shown). VLPs displaying full-length mature ApoC3 (SEQ ID NO:3) were constructed by genetically inserting the ApoC3 gene at the C-terminal of a single-chain dimer version of the AP205 coat protein. In this conformation, each recombinant VLP displays 90 copies of the ApoC3 protein.

While exemplified below in the context of model, exemplary embodiments in which the ApoC3 peptide includes the amino acid sequence of SEQ ID NO:1 and/or SEQ ID NO:2, the immunogen can include any suitable ApoC3-derived peptide. As used herein, an ApoC3-derived peptide includes an immunogenic fragment of SEQ ID NO:3 (the full-length, mature human ApoC3 polypeptide), SEQ ID NO:4 (the human ApoC3 preprotein [symbol] ApoC3 peptides include, but are not limited to, a peptide fragment o [symbol] ID NO:1 or TKTAKDALSS, amino acids 40-49 of SEQ ID NO:4), a peptide fragment of ApoC2 helix 5 (e.g., SEQ ID NO:2), a fragment of the lipid binding region (SSLKDYWSTVKDKFSEFWDLDPEVRPTSAVAA, amino acids 68-99 of SEQ ID NO:4), or the full-length mature ApoC3 polypeptide (SEQ ID NO:3). Alternatively, the ApoC3 peptide can be a fragment of helix 1 (e.g., LLSFMQGYMKH, amino acids 28-38 of SEQ ID NO:4), a fragment of helix 3 (e.g., amino acids 53-63 of SEQ ID NO:4), a fragment of helix 4 (e.g., amino acids 66-74 of SEQ ID NO:4), or a fragment of helix 6 (e.g., amino acids 94-99 of SEQ ID NO:4). Finally, in certain embodiments, the ApoC3 peptide may be a fragment of the signal peptide (MQPRVLLVVALLALLASARA, amino acids 1-20 of SEQ ID NO:4) or the N-terminal domain (SEAEDAS, amino acids 21-27 of SEQ ID NO:4).

In some embodiments, the ApoC3 peptide can be based on an immunogenic fragment of SEQ ID NO:3 or SEQ ID NO:4, but include one or more modifications to the amino acid sequence of the base polypeptide fragment. Any modifications to the amino acid sequence will not abrogate the immunogenicity of the peptide fragment. Exemplary amino acids modifications include conservative amino acid substitutions, an addition to the C-terminus of the peptide fragment, and/or an addition to the N-terminus of the peptide fragment.

A conservative substitution for an amino acid in an ApoC3 peptide may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. Likewise, an ApoC3-derived peptide can include a deletion or an ad [symbol] contiguous or noncontiguous amino acids that do not eliminate a fu [symbol] ApoC3 peptide are also contemplated.

An ApoC3 peptide also can be designed to provide additional sequences, such as, for example, the addition of coding sequences for added C-terminal or N-terminal amino acids that, can, for example, facilitate purification by trapping on columns or use of antibodies. Such tags include, for example, histidine-rich tags that allow purification of polypeptides on nickel columns. Such gene modification techniques and suitable additional sequences are well known in the molecular biology arts.

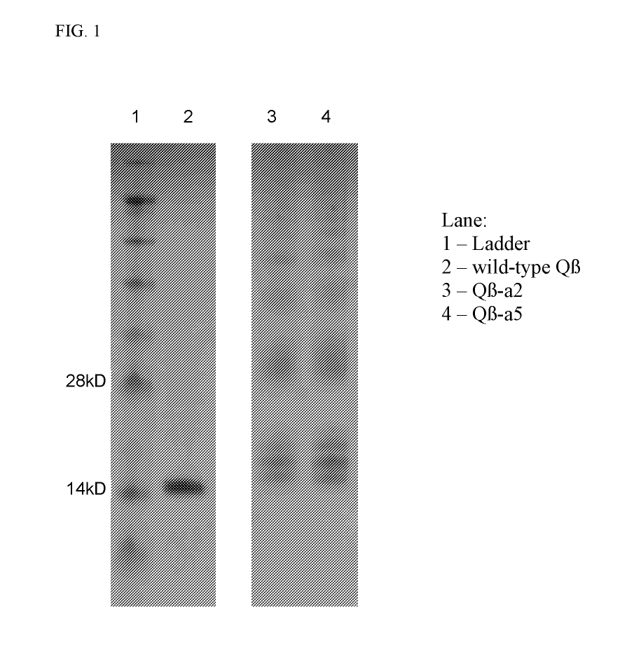

Mice were immunized intramuscularly with 5 μg of Qβ-a5, Qβ-a2, or AP205-ApoC3 VLPs. Mice were given two immunizations at an interval of 3 weeks. As controls, groups of mice were immunized with 5 μg of either wild-type Qβ or wild-type AP205 VLPs. Plasma was collected prior to immunization and two weeks after the booster immunization. Antibody titers against ApoC3 and the selected ApoC3 peptides (a2 and a5) were assessed by end-point dilution ELISA. As shown in FIG. 1, mice immunized with VLPs displaying full-length ApoC3 elicited antibody responses against ApoC3 as well as the two ApoC3 peptides. Mice immunized with VLPs displaying the ApoC3 peptides elicited antibody responses against the respective peptide.

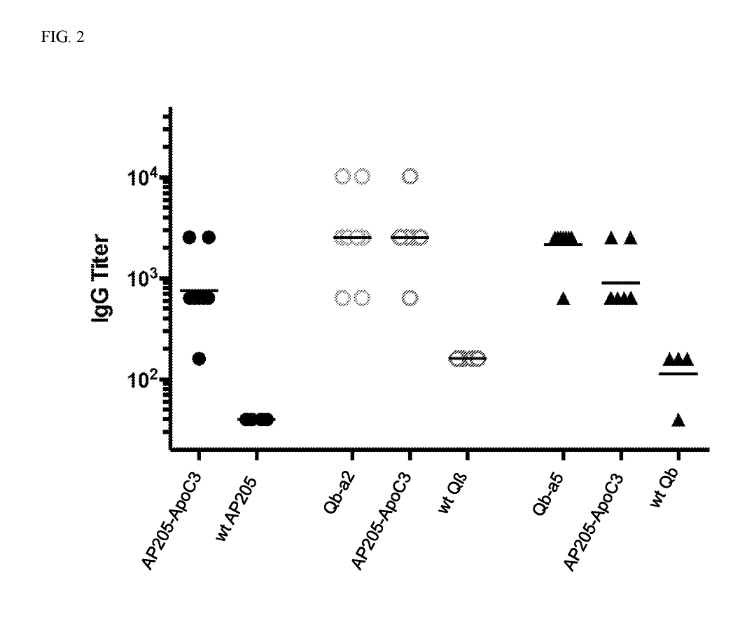

To assess the effects on lipids, total cholesterol levels and triglyceride levels were measured in the vaccinated mice and compared those levels to pre-bleed samples (FIG. 2). Mice immunized with Qβ-a5 and AP205-ApoC3 had reduced triglyceride levels (40% and 30%, respectively) relative to pre-bleed levels, whereas there was no reduction in the groups immunized with Qβ-a2 or wild-type VLPs. No reduction in total cholesterol in any of the groups was detected.

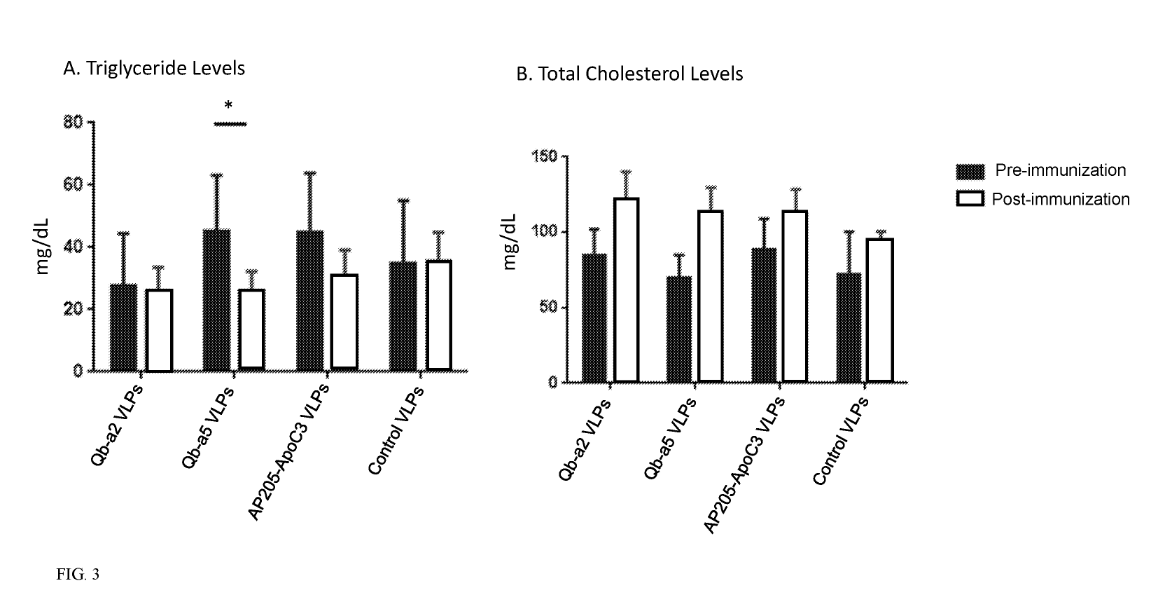

The presence of autoantibodies against the serum protein PCSK9 can increase soluble PCSK9 levels in the plasma. To assess the effects of vaccination on ApoC3, serum levels were measured by ELISA (FIG. 3). Elevated ApoC3 levels (relative to controls) were detected in mice immunized with Qβ-a5 VLPs, but not Qβ-a2 or AP205-ApoC3 VLPs.

Thus, this disclosure describes an immunogen that includes an antigenic ApoC3 peptide linked to a bacteriophage virus like particle (VLP) Qβ immunogenic carrier. In some embodiments, the antigenic ApoC3 peptide includes the amino acid sequence STVKDKFSEF (SEQ ID NO:2).

The immunogen can be a formulated into a pharmaceutical [symbol] pharmaceutically acceptable carrier. As used herein, “carrier” inclu [symbol] medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the immunogen without causing any undesirable biological effects or interacting in a deleterious manner with any of the other components of the pharmaceutical composition in which it is contained.

The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, the immunogen may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a preservative, an agent that promotes thermostability, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the [symbol] with a carrier that constitutes one or more accessory ingredients. In [symbol] be prepared by uniformly and/or intimately bringing the immunogen into association with a liquid carrier, a finely divided solid carrier, or both, and then, if necessary, shaping the product into the desired formulations.

Thus, in another aspect, this disclosure describes a method of treating a subject having, or at risk of having, dyslipidemia and/or a condition associated with or caused by dyslipidemia (e.g., pancreatitis is associated with high triglyceride levels). Generally, the method includes administering to the subject an effective amount of a pharmaceutical composition that includes the immunogen. As used herein, the term “treat” or variations thereof refer to reducing, limiting progression, ameliorating, or resolving, to any extent, the symptoms or signs of dyslipidemia. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after dyslipidemia manifests in a subject, while “prophylactic” treatment is initiated before dyslipidemia manifests in a subject. As used herein, the term “symptom” refers to any subjective evidence of disease or of a patient's condition, while the term “sign” or “clinical sign” refers to an objective physical finding relating to a particular condition capable of being found by one other than the patient. As used herein, the term “ameliorate” refers to any reduction in the extent, severity, frequency, and/or likelihood of a symptom or clinical sign characteristic of dyslipidemia.

Treating dyslipidemia can be prophylactic or, alternatively, can be initiated after the subject exhibits one or more symptoms or clinical signs of dyslipidemia. Treatment that is prophylactic—e.g., initiated before a subject manifests a symptom or clinical sign of dyslipidemia—is referred to herein as treatment of a subject that is “at risk” of having dyslipidemia. As used herein, the term “at risk” refers to a subject that may or may not actually possess the described risk. Thus, for example, a subject “at risk” of developing dyslipidemia is a subject possessing one or more risk factors associated with dyslipidemia such as, for example, genetic predisposition, ancestry, age, sex, geographical location, lifestyle, or medical history. For example, dyslipidemia (i.e., hypercholesterolemia) is defined as elevated total cholesterol (>240 mg/dL), elevated LDL-C (>160 mg/dL), or low HDL-C (<40 [symbol] levels are defined as 150-199 mg/dL (borderline high), 200-499 m [symbol] mg/dL (very high).

Accordingly, administration of a composition can be performed before, during, or after the subject first exhibits a symptom or clinical sign of dyslipidemia and/or condition associated with or caused by dyslipidemia. Treatment initiated before the subject first exhibits a symptom or clinical sign associated with dyslipidemia may result in decreasing the likelihood that the subject experiences clinical evidence of dyslipidemia compared to a subject to whom the composition is not administered, decreasing the severity of symptoms and/or clinical signs of dyslipidemia, and/or completely resolving dyslipidemia. Treatment initiated after the subject first exhibits a symptom or clinical sign associated with dyslipidemia may result in decreasing the severity of symptoms and/or clinical signs of dyslipidemia compared to a subject to whom the composition is not administered, and/or completely resolving dyslipidemia.

Thus, the method includes administering an effective amount of the composition to a subject having, or at risk of having, dyslipidemia and/or condition associated with or caused by dyslipidemia. In this aspect, an “effective amount” is an amount effective to reduce, limit progression, ameliorate, or resolve, to any extent, a symptom or clinical sign related to dyslipidemia and/or condition associated with or caused by dyslipidemia.

The amount of the immunogen administered can vary depending on various factors including, but not limited to, the weight, physical condition, and/or age of the subject, and/or the route of administration. Thus, the absolute weight of immunogen included in a given unit dosage form can vary widely, and depends upon factors such as the species, age, weight and physical condition of the subject, and/or the method of administration. Accordingly, it is not practical to set forth generally the amount that constitutes an amount of immunogen effective for all possible applications. Those of ordinary skill in the art, however, can readily determine the appropriate amount with due consideration of such factors.

In some embodiments, the method can include administering sufficient immunogen to provide a minimum dose of at least 10 μg to the subject such as, for example, a dose of at least 25 μg, at least 50 μg, at least 75 μg, at least 100 μg, at least 150 μg, at least 200 μg, or at least 250 μg. In some embodiments, the method can include administering sufficient immunogen to provide a maximum dose of no more than 1000 μg to the subject such as, for example, a dose of no more than 500 μg, no more than 400 μg, no more than 300 μg, n [symbol] than 100 μg, or no more than 50 μg. In one exemplary embodiment, [symbol] administering the immunogen to a subject at a dose of 100 μg.

In some embodiments, the dose can be expressed as a range having endpoints defined by any minimum dose listed above and any maximum dose listed above that is greater than the minimum dose. For example, the method can include administering the immunogen to a subject at a dose of from 10 μg to 500 μg such as, for example, a dose of from 50 μg to 300 μg.

The frequency of administration can vary depending on any of a variety of factors, e.g., severity of the symptoms, degree of immunoprotection desired, whether the composition is used for prophylactic or therapeutic purposes, etc. For example, in some embodiments, the immunogen may be administered once every six months, once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). In some embodiments, the immunogen may be administered from two to four times per year such as, for example, from two to three times per year.

When the immunogen is used prophylactically, it can be administered for both priming and boosting doses. Boosting doses can be administered at regular or pre-determined intervals or may be administered as needed—e.g., when the level of circulating antibody falls below a desired level. Boosting doses may include the antigenic ApoC3 peptide in the absence of the immunogenic carrier molecule. Such booster constructs may include an alternative immunogenic carrier or may be in the absence of any carrier. Such booster compositions may be formulated either with or without adjuvant.

The duration of administration of an antigenic apoC3 peptide—e.g., the period of time over which an antigenic ApoC3 peptide is administered to a subject—can vary, depending on any of a variety of factors such as, for example, patient response, etc. For example, an antigenic ApoC3 peptide can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments, the VLP may be co-administered wit [symbol] dyslipidemia therapeutic agent. As used herein, “co-administered” r [symbol] of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another.

In some embodiments, the additional dyslipidemia therapeutic agent comprises a statin, a PCSK9 inhibitor, a bile acid sequestrant, niacin, a fibric acid derivative, and a long chain alpha, omego-dicarboxylic acid.

While described in the context of an exemplary embodiment in which the VLP includes Qβ or AP205, the VLP can include any suitable virus-like particle. As used herein, the term “virus-like particle” refers to a structure resembling a virus particle but is nonpathogenic. In general, a virus-like particle lacks at least part of the viral genome. A virus-like particle can be recombinant—i.e., can contain nucleic acid distinct from its native genome. One embodiment of a virus-like particle is a viral capsid such as the viral capsid of a corresponding virus, bacteriophage, or RNA-phage.

Examples of VLPs suitable as immunogenic carriers in the context of the present invention include, but are not limited to, VLPs of Qβ, MS2, PP7, AP205 and other bacteriophage coat proteins, the capsid and core proteins of Hepatitis B virus, measles virus, Sindbis virus, rotavirus, foot-and-mouth-disease virus, Norwalk virus, the retroviral GAG protein, the retrotransposon Ty protein pi, the surface protein of Hepatitis B virus, the core protein of Hepatitis B virus, human papilloma virus, human polyoma virus, RNA phages, Ty, frphage, GA-phage, AP 205 -phage and, in particular, Qβ-phage, Cowpea chlorotic mottle virus, cowpea mosaic virus, human papilloma viruses (HPV), bovine papilloma vi [symbol] parvoviruses such as B19, porcine (PPV) and canine (CPV) parvovi [symbol] Norwalk virus, rabbit hemorrhagic disease virus [RHDV]), animal hepadnavirus core Antigen VLPs, filamentous/rod-shaped plant viruses, including but not limited to Tobacco Mosaic Virus (TMV), Potato Virus X (PVX), Papaya Mosaic Virus (PapMV), Alfalfa Mosaic Virus (AIMV), and Johnson Grass Mosaic Virus (JGMV), insect viruses such as flock house virus (FHV) and tetraviruses, polyomaviruses such as Murine Polyomavirus (MPyV), Murine Pneumotropic Virus (MPtV), BK virus (BKV), and JC virus (JCV). Methods of constructing an immunogen that includes a VLP are described in International Patent Application Publication No. WO/2015/123291.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

This example describes the conjugation of ApoC3 peptide T [symbol] NO:1) or STVKDKFSEF (SEQ ID NO:2) to Qβ bacteriophage VLPs using the bifunctional cross-linker SMPH (Pierce Endogen, IL), described previously (Hunter et al., 2011, Vaccine 29(28):4584-4592). As depicted in FIG. 1, this technique links the C-terminal cysteine on the PCSK9 peptide to exposed surface lysine residues on the coat protein of Qβ. Conjugation efficiency was monitored by SDS-PAGE analysis. Each Qβ-ApoC3-peptide particle displays an average of ˜300 peptides per VLP.

### Example 2

This example describes the mean end-point dilution titers in groups of eight balb/c mice immunized with VLPs displaying full-length ApoC3 or ApoC3 peptides. Groups of five mice were immunized with 5 μg of the indicated VLPs two times at a three-week interval. Approximately two weeks after the boost, sera were tested for antibody responses against either full-length ApoC3 (filled circles), the a2 peptide (open circles), or the a5 peptide (triangles) by ELISA. Each data point indicates an individual mouse. These data demonstrate that vaccination with the ApoC3-displaying VLPs elicit high titer IgG antibody responses.

### Example 3

This example (shown in FIG. 3) describes testing the analysis of plasma total cholesterol and triglyceride levels from mice immunized as described in Example 2. Groups of eight balb/c mice were immunized with AP205-ApoC3, Qβ-a2, Qβ-a5, or, as a control, wild-type VLPs. At baseline (black bars) and at approximately two weeks after the second immunization (white bars) plasma lipids levels were measured. Bars show the mean values plus the standard deviation. * indicates p<0.05, by 2-way ANOVA. Vaccination with AP205-ApoC3 and Qβ-a5 resulted in a 30-40% decrease in triglyceride levels, but not effect on total cholesterol levels.

### Example 4

This example describes testing the levels of serum-associated ApoC3 protein in mice vaccinated AP205-ApoC3, Qβ-a2, Qβ-a5, or, as a control, wild-type VLPs, as determined by mouse ApoC3 ELISA (Abnova). Each data point represents an individual mouse. * indicates p<0.05 by Dunnett's multiple comparisons test. After vaccination, the [symbol] immunized with Qβ-a5 were significantly higher than in controls.

[symbol]

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ ID NO: 1-ApoC3 amino acids 22-30 (a2 peptide)
TAKDALSSV
SEQ ID NO: 2-ApoC3 amino acids 55-64 (a5 peptide)
STVKDKFSEF
SEQ ID NO: 3-Full length (mature) ApoC3
SEAEDASLLS FMQGYMKHAT KTAKDALSSV QESQVAQQAR
GWVTDGFSSL KDYWSTVKDK FSEFWDLDPE VRPTSAVAA
SEQ ID NO: 4-Human ApoC3 preprotein sequence:
MQPRVLLVVA LLALLASARA SEAEDASLLS FMQGYMKHAT
KTAKDALSSV QESQVAQQAR GWVTDGFSSL KDYWSTVKDK
FSEFWDLDPE VRPTSAVAA

1. An immunogen comprising an antigenic ApoC3 peptide linked to a bacteriophage virus like particle (VLP) immunogenic carrier.

2. The immunogen of claim 1 wherein the antigenic ApoC3 peptide comprises the amino acid sequence STVKDKFSEF (SEQ ID NO:2).

3. The immunogen of claim 1 wherein the VLP immunogenic carrier comprises Qβ.

4. A composition comprising the immunogen of claim 1.

5. An immunogenic composition comprising:
the immunogen of claim 1;
an adjuvant.

6. A method of treating a subject having, or at risk of having, dyslipidemia, the method comprising:
administering an effective amount of the immunogen of claim 1 to the subject.

7. The method of claim 6 comprising co-administering to the subject at least one additional dyslipidemia therapeutic agent.

8. The method of claim 7 wherein the additional dyslipidemia therapeutic agent comprises a statin, a bile acid sequestrant, niacin, a fabric acid derivative, and a long chain alpha, omega-dicarboxylic acid.

9. The method of claim 6 wherein the subject further has, or is at risk of having, pancreatitis.

10. A nucleic acid encoding the immunogen of claim 1.

11. An expression vector comprising the nucleic acid of claim 10.

12. A host cell comprising the expression vector of claim 11.


SEQUENCE LISTING (4 sequences)

SEQ ID NO: 1 - protein, 9 aa, Homo sapiens
TAKDALSSV

SEQ ID NO: 2 - protein, 10 aa, Homo sapiens
STVKDKFSEF

SEQ ID NO: 3 - protein, 79 aa, Homo sapiens
SEAEDASLLSFMQGYMKHATKTAKDALSSVQESQVAQQARGWVTDGFSSLKDYWSTVKDKFSEFWDLDPEVRPTSAVAA

SEQ ID NO: 4 - protein, 99 aa, Homo sapiens
MQPRVLLVVALLALLASARASEAEDASLLSFMQGYMKHATKTAKDALSSVQESQVAQQARGWVTDGFSSLKDYWSTVKDKFSEFWDLDPEVRPTSAVAA